(19) United States
(12) Patent Application Publication
(10) Pub. No.: US 2022/0062605 A1
(43) Pub. Date: Mar. 3, 2022

(54) Applicator

(71) Applicant: LTS Lohmann Therapie-Systeme AG, Andemach (DE)

(72) Inventors: Stefan Erlhofer, Kempenich (DE); Sebastian Scherr, Neuhäusel (DE)

(21) Appl. No.: 17/423,509

(22) PCT Filed: Nov. 27, 2019
(86) PCT No.: PCT/EP2019/082699

§ 371 Date: Jul. 16, 2021

(30) Foreign Application Priority Data
Jan. 17, 2019 (DE) 10 2019 200 563.2

(51) Int. Cl. A61M 37/00

(52) U.S. Cl. CPC A61M 37/0015; A61M 2037/0023

(57) ABSTRACT

The invention relates to an applicator for application of a microarray, comprising a housing (10) in which a plunger (20) is arranged such that it can move linearly. A retainer element (38) is also provided for receiving a microarray (28). A pressure element (34) makes it possible for the plunger (20) to be displaced in order for it to act upon a microarray (28) receive by the retainer element (38). An actuation element (44) allows the plunger (20) to be held in an internal position, and this can be released by actuating said actuation element (44).

### CROSS-REFERENCE TO RELATED APPLICATIONS

This application is the United States national phase of International Application No. PCT/EP2019/082699 filed Nov. 27, 2019, and claims priority to German Patent Application No. 102019200563.2 filed Jan. 17, 2019, the disclosures of which are hereby incorporated by reference in their entirety.

### BACKGROUND OF THE DISCLOSURE

### Field of the Disclosure

The disclosure relates to an applicator for the application of a microarray.

### Description of Related Art

Microarrays comprise a plurality of microneedles which is usually arranged on a carrier element such as a patch, a plaster or the like, or is connected to a carrier element. Such microarrays comprise a large number of microneedles, e.g., 500-600 needles per cm². The needles are short in length so that upon pushing the microneedles into the skin of a patient, the needles will penetrate the skin of a patient only to an extent that nerves and blood vessels preferably do not come into contact with any needle tip. The microneedles include an active substance or a drug. The corresponding active substance may be applied on a surface of the needle or be provided in the needles. It is preferred that the needles are made of a material that dissolves in the skin of a patient.

When applying microarrays into human skin, there is a problem that the insertion of the microneedles into the skin must be reproducible so as to guarantee in particular a reliable drug delivery. Further, the insertion of the microneedles must be independent of the user or patient, since a reproducibility would otherwise not be guaranteed. In particular, the insertion of the microneedles should also be independent of the skin texture so that a certain penetration depth is guaranteed at all times.

### SUMMARY OF THE DISCLOSURE

It is an object of the disclosure to provide an applicator for the application of a microarray, which improves the reproducibility of the insertion of the microarray.

The object is achieved according to the disclosure by an applicator having the features of claim 1.

The applicator according to the present disclosure for the application of a microarray comprises a housing in which a plunger is arranged for linear movement. Further, a retainer element for receiving a microarray is provided, in particular connected with the housing or arranged thereon. Here, the microarray may be received indirectly or directly by the retainer element. In particular, the retainer element may receive a carrier element, such as a patch or the like, which is connected with the microarray or carries the microarray. The applicator according to the present disclosure further comprises a pressure element for the displacement of the plunger.

By a corresponding displacement of the plunger, it is possible to act on the microarray retained by the retainer element. It is preferred that the pressure element applies in particular a short-term force on the microarray so that the needles are pressed into the skin of the patient. The applicator according to the present disclosure further comprises an actuation element for the actuation of the plunger. The actuation element indirectly or directly maintains the plunger in an inner or retracted position, i.e., in a position prior to the insertion of the microarray into the skin of the patient. Here, the actuation element may act either directly on the plunger, be connected with the same, e.g., in a form-fitting manner, or also act on the pressure element. By an in particular manual actuation of the actuation element, a release is effected so that the plunger is released for displacement. By an in particular sudden or impulsive movement of the plunger, the plunger presses the microarray into the skin with a corresponding velocity. Thereby, an in particular clearly defined force acts on the microarray so that a good reproducibility of the insertion of the microarray into the skin of the patient is realized.

In a preferred development, the retainer element has a receiving surface for arranging a carrier element connected with the microarray, such as a patch, a plaster or the like. The carrier element may, for example, be arranged by adhering the same on the receiving surface. Preferably, the in particular planar receiving surface at least has the shape of a partial ring and, as is particularly preferred, the shape of a complete ring.

The housing of the applicator preferably comprises an exit opening through which the plunger may exit. In a preferred embodiment the plunger is pushed out through the exit opening by the pressure element or acts on the microarray in the region of the exit opening. Furthermore, it is particularly preferred that the plunger and/or the exit opening for the plunger are surrounded at least in part, in particular completely, by the receiving surface. Here, the plunger is surrounded by the receiving surface in particular in its outer position, i.e., in the position in which the microarray is or was pressed into the skin by the plunger.

Preferably, the pressure element is prestressed and/or compressed in the inner position of the plunger. It is particularly preferred that the pressure element is a spring, such as a helical spring, a pneumatic or hydraulic chamber or combinations thereof. The pressure element may, e.g., also comprise an elastic element, such as a compressible plastic material, acting on the plunger.

The pressure element is preferably arranged on a rear side of the plunger opposite the exit opening. The pressure element may, e.g., also laterally and tat other positions and microarray cooperate with the plunger such that, upon actuation or triggering of the plunger, a linear movement of the plunger towards the microarray occurs in order to push the same into the skin of the patient. For realizing as simple a structure as possible and a good force transmission, it is preferred that the pressure element is arranged on the rear side of the plunger.

In another particularly preferred embodiment, the actuation element cooperates directly with the plunger. Here, an actuation element is particularly preferred that can be manually operated by a patient, doctor or the like. It is further preferred that the actuation element for retaining the plunger in the inner position is form-fittingly connected with the plunger and thus retains the plunger in its inner position. As an alternative or in addition, the actuation element may also be connected with the pressure element, whereby an actuation of the actuation element causes the pressure element to expand or relax, since it causes an actuation or a linear displacement of the plunger. Here, the plunger does not necessarily have to be retained in an inner position by the actuation element. This is preferred essentially when the pressure element is retained in a controlled position by the plunger itself and no separate triggering of the pressure element is effected.

It is preferred that the plunger has a recess or a protrusion, which cooperates with the actuation element. In particular, the actuation element or a protrusion on the actuation element may extend into the recess. By actuating, in particular by displacing the actuation element, the actuation element is moved out from the recess so that the plunger is released and can be moved linearly by the pressure element. Here, it is preferred that the actuation element is displaced and/or twisted to release the plunger. Preferably, the actuation element is arranged in a receptacle of the housing. This may be a recess, a slot-shaped opening or the like.

In a particularly preferred development of the disclosure, the housing comprises a fixation element and/or is connected with a fixation element. The fixation element serves to fix the applicator on a patient's skin. This has the advantage that after the actuation of the actuation element and thus, after the displacement of the plunger for pressing the microarray, the microarray can be maintained in the inserted position in a simple manner. For example, a vacuum element could be provided as the fixation element. Similar to a suction cup, the same would be used to create a vacuum so that the applicator sucks itself to the skin of a patient. The vacuum could be created by pressing the applicator onto the skin of a patient. In addition, or instead of this creation of a vacuum by pressing, the creation of a vacuum may also be effected through the pressure element. The pressure element could be configured such that upon actuation of the plunger and the insertion of the microarray into the skin, a vacuum is created at the same time by which the applicator is sucked to the skin of a patient.

It is preferred that the fixation element comprises a contact surface which is in particular planar. The contact surface at least partly surrounds the receiving surface that serves to arrange the carrier element for the microarray thereon. Preferably, the contact surface is shaped as a partial ring and in particular as a complete ring, in particular corresponding to the contact shape. It is furthermore particularly preferred that the contact surface is provided with an adhesive layer to fix the applicator or the fixation element on the skin of a patient. The contact surface provided with an adhesive layer is preferably provided with a peelable or removable protective film or the like.

After the application of the microarray into the skin of a patient by the plunger, it is possible, according to a preferred embodiment, to maintain the microarray in the inserted position by the pressure element. This is possible, e.g., by the pressure element exerting a residual force.

The disclosure will be explained in more detail hereunder with reference to a preferred embodiment and to the accompanying drawings.

### BRIEF DESCRIPTION OF THE DRAWINGS

In the Figures:

FIG. 1 is a schematic perspective top plan view of an embodiment of the applicator according to the present disclosure, and

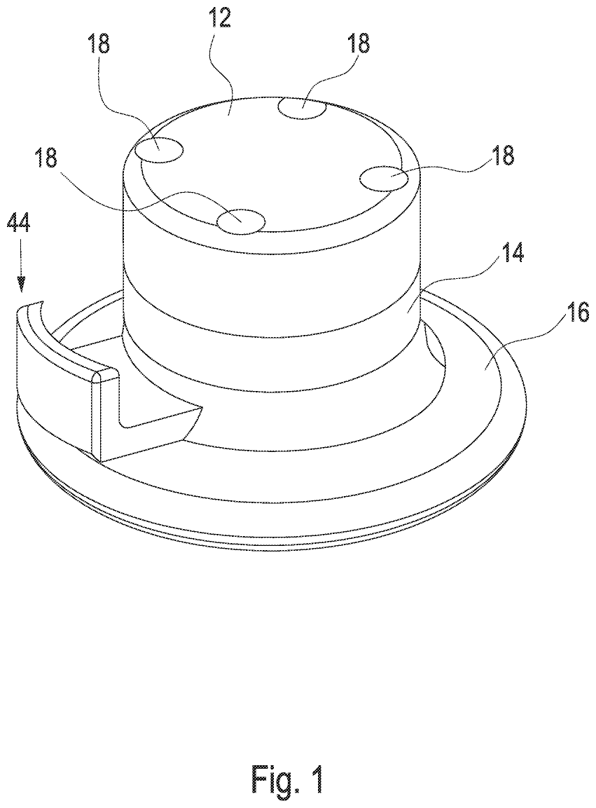

FIG. 2 is a schematic sectional view illustrating the applicator of FIG. 1.

### DETAILED DESCRIPTION

The applicator comprises a housing 10. In the embodiment illustrated, is composed of a cover 12, an intermediate 14 and a base element 16, wherein the intermediate element 14 and the base element 16 can be formed integrally. In the embodiment illustrated, the cover element 12 is fixed by pins or screws 18, while a fixation by gluing, a snap connection, a screw connection or the like could be made as well. A plunger 20 is arranged within the housing 10. In the embodiment illustrated, the plunger 20 is designed to be rotationally symmetrical with respect to a central axis 22. The plunger 20 comprises a plunger element 26 on a front side 24 directed towards the skin of the patient when the applicator is used. The plunger element 26 has a cross-section corresponding to the dimensions of a microarray 28. The microarray 28 may, e.g., be round or square or also have another geometric shape.

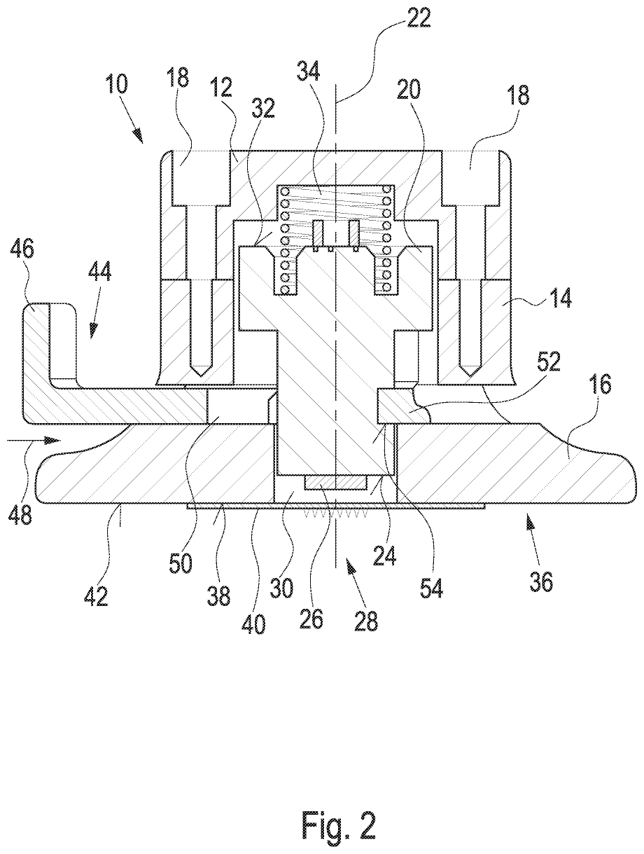

A pressure element 34 is arranged on a rear side 32 of the plunger 20 opposite the front side 24 or an exit opening 30. In the embodiment illustrated, the pressure element 34 is a helical spring. In the inner position of the plunger 20 illustrated in FIG. 2, the compression spring 34 is compressed, the spring being supported on an inner side of the housing cover 12.

The base element, which in the embodiment illustrated is also rotationally symmetric with respect to the central axis 22, has a planar lower side 36. The planar lower side 36 has an inner annular region 38 serving as a retaining element and as a receiving surface for the patch 40 that carries or is connected with the microarray 28. Here, the patch 40 can be arranged on the receiving surface 38 in particular by adhesion. The annular receiving surface 38 is surrounded by a likewise annular contact surface 42. The contact surface 42 serves to make contact with the skin of a patient. Preferably, the contact surface 42 is also provided with an adhesive layer which may be covered by a removable film. Here, the lower side 36 may also be formed in a stepped shape so that in particular the receiving surface 38 is formed as a recess with respect to the contact surface 40 so that the patch 40 together with the microarray 28 does, in particular, not protrude beyond the contact surface 42.

Furthermore, the applicator has an actuation element 44, in the embodiment illustrated, the actuation element 44 is essentially L-shaped and has a protrusion 46 by which the actuation element can be operated manually. In particular, the actuation element can be displaced inward in the direction of an arrow 48 from the position illustrated in FIG. 2. By displacing the actuation element inward, an opening or recess 50 of the actuation element is displaced to the right in FIG. 2 from a recess 54 provided in the plunger 20. Thereby, the plunger 20 is released and can thereafter be displaced linearly downward through the opening 50 in FIG. 2. As soon as the actuation element 44 is pushed inward in the direction of the arrow 48 and the plunger is released, the latter is displaced immediately downward in FIG. 2 towards the microarray 28. This is effected by the compressed spring 34. A linear displacement of the plunger 20 causes an in particular sudden insertion of the microarray 28 into the skin of a patient, not illustrated. The microarray 28 is maintained in the inserted position in the skin by the residual force exerted by the spring 34. Since, in the embodiment illustrated, the applicator is configured as an adhesive surface and comprises a fixation element 42, the applicator is also retained on the skin of the patient. By providing a fixation element, as provided according to the disclosure in a preferred embodiment, it is possible to maintain the microarray in the position inserted in the skin of a patient and to avoid the microarray from being pushed out, in particular because of the elasticity of the skin.

1. An applicator for the application of a microarray comprising:
a housing in which a plunger is arranged for linear displacement therein,
a retainer element for receiving the microarray,
a pressure element for the displacement of the plunger in order to act on a microarray received by the retainer element, and
an actuation element retaining the plunger in an inner position and releases the plunger for displacement by actuation of the actuation element.

2. The applicator according to claim 1, wherein the retaining element comprises a receiving surface for arranging thereon a carrier element, in particular a patch, connected with the microarray, said receiving surface being shaped at least as a partial ring, in particular a complete ring.

3. The application according to claim 1, wherein the housing has an exit opening for the plunger.

4. The applicator according to claim 2, wherein the receiving surface surrounds the plunger and/or the exit opening, in particular completely.

5. The applicator according to claim 1, wherein the pressure element is prestressed and/or compressed in the inner position of the plunger.

6. The applicator according to claim 1, wherein the pressure element comprises a spring element.

7. The applicator according to claim 3, wherein the pressure element is arranged on a rear side of the plunger opposite the exit opening.

8. The applicator according to claim 1, wherein the actuation element cooperates directly with the plunger, the element in particular being connectable with the plunger in a form-fitting manner to maintain the plunger in the inner position.

9. The applicator according to claim 8, wherein the plunger comprises a recess or a protrusion, which cooperates with the actuation element.

10. The applicator according to claim 1, wherein the actuation element can be displaced and/or twisted to release the plunger.

11. The applicator according to claim 1, wherein the actuation element is arranged in a recess of the housing.

12. The applicator according to claim 1, wherein the housing comprises a fixation element for fixing the applicator on the skin of a patient.

13. The applicator according to claim 12, wherein the fixation element comprises a contact surface which preferably surrounds the receiving surface at least in a partial ring shape or a complete ring shape.

14. The applicator according to claim 13, wherein the contact surface comprises an adhesive layer which is preferably provided with a removable protective film.
